(19) United States
(12) Patent Application Publication
(10) Pub. No.: US 2017/0042444 A1
(43) Pub. Date: Feb. 16, 2017

(54) SYSTEMS AND METHODS FOR OBJECTIVELY DETERMINING HEARING THRESHOLDS

(71) Applicant: HEAR IP PTY LTD, The University of Melbourne, Victoria (AU)

(72) Inventors: Fabrice BARDY, Macquarie University, New South Wales (AU); Harvey DILLON, Macquarie University, New South Wales (AU); Bram VAN DUN, Macquarie University, New South Wales (AU)

(21) Appl. No.: 15/305,676

(22) PCT Filed: Apr. 22, 2015
(86) PCT No.: PCT/AU2015/050187

§ 371 Date: Oct. 21, 2016

(30) Foreign Application Priority Data
Apr. 23, 2014 (AU) 2014901474

(51) Int. Cl. A61B 5/0484; A61B 5/12

(52) U.S. Cl. CPC A61B 5/04845; A61B 5/125; A61B 5/7235

(57) ABSTRACT

A system and method for determining hearing thresholds of people. The system and methods include the steps of presenting an auditory stimulus of a duration of less than one second to the person; spreading the power of the stimulus across a frequency range in alternating regions of high and low intensity; including in the stimulus at least two regions of high intensity; and measuring the transient evoked electrical response of the brain of the person.

### TECHNICAL FIELD

The present invention relates to systems and methods for objectively determining hearing thresholds.

### BACKGROUND TO THE INVENTION

In audiology, a hearing threshold is defined as being the softest sounds that can be detected a defined proportion of the time, for stimuli that have their energy within a defined range of frequencies. Hearing thresholds are an important tool used in assessing the hearing of a person. Hearing threshold are estimated during hearing testing either behaviourally or objectively.

“Behavioural” hearing tests require the participant to reliably demonstrate a change in behaviour when a test sound is heard such as by pressing a button. For adults and older children, the most commonly used forms of behavioural tests are pure tone audiometry and speech discrimination testing. These two measures provide information on the degree and nature of any hearing loss.

Pure tone audiometry involves listening to sounds across a range of pitches, or notes, and responding when the stimulus is heard. The examiner systematically finds the softest sounds the participant can hear across a range of frequencies and determines the hearing thresholds—the softest sounds one can hear across the range of frequencies that are important for speech understanding. The hearing thresholds are plotted on a graph called an audiogram.

“Objective” hearing tests require only passive co-operation from the participant. These tests may be performed to gain insight into the potential causes of a hearing abnormality and are sometimes carried out by way of electrophysiological testing. Objective hearing threshold estimation is convenient for patients who are not able to provide behavioural feedback, such as young children or adults who cannot or will not subjectively cooperate with testing.

In the absence of sensory stimulation, the central nervous system generates spontaneous random neuro-electrical activity which can be recorded with sensors (electrodes) positioned on the scalp. When sound is presented to the patient, neural activity within the auditory system is generated. This activity is however minute compared with ongoing brain activities.

Electrodes are applied to the patient's scalp and a pre-determined acoustic stimulus is presented to the patient. The signals evoked in the brain by the stimulus are measured. Because the evoked brain signals measureable on the scalp are smaller than other voltages also present on the scalp arising from the combination of numerous other sources within the brain, muscle activity, and surrounding electrical apparatus, the stimulus is presented many times. The measured waveforms are averaged in a manner synchronised with the stimulus, so that the signal evoked by the stimulus emerges from the other voltages present. It can take from a few tens of seconds up to many minutes for the wanted signal to emerge as something unambiguously related to the stimulus.

One example of electophysiological testing is measuring cortical auditory evoked potentials (CAEPs) which reflect the activation at the level of the central auditory system in the supratemporal auditory cortex. Their recording relies on the averaging of synchronous far-field neuronal potentials evoked by auditory stimuli presented multiple times. For awake adults, the P1-N1-P2 complex generated in the time window 50-200 ms after onset of an acoustical stimulus is the response of interest. CAEPs are appreciated because they can be elicited by highly frequency-specific stimuli. In addition, CAEP testing is more preferable than brainstem testing where the subject needs to be asleep. This is often a difficult condition to achieve at ages of 6 months and older.

During a hearing threshold test many different stimuli are presented many times which can result in a long duration of testing involving both the audiologist and the test subject. It would be advantageous to reduce the time required to conduct objective hearing threshold testing.

### SUMMARY OF THE INVENTION

In a first aspect, the present invention provides a method of determining a hearing threshold of a person including the steps of: presenting an auditory stimulus of a duration of less than one second to the person; wherein the power of the stimulus is spread across a frequency range in alternating regions of high and low intensity: and wherein the stimulus includes at least two regions of high intensity; and measuring the transient evoked electrical response of the brain of the person,

The alternating regions of high and low intensity may be centered around a target frequency of interest.

The stimulus may include three or more regions of high intensity.

The regions of high intensity may include narrow bands.

The regions of high intensity may include pure tones.

The stimulus may have a duration of less than 200 ms.

The stimulus may have a duration of less than 100 ms.

The stimulus may have a duration of approximately 50 ms.

In a second aspect, the present invention provides a system for determining hearing thresholds of a person including: means for presenting an auditory stimulus of a duration of less than one second to the person; wherein the power of the stimulus is spread across a frequency range in alternating regions of high and low intensity; and wherein the stimulus includes at least two regions of high intensity; and means for measuring the transient evoked electrical response of the brain of the person.

### BRIEF DESCRIPTION OF THE DRAWINGS

An embodiment of the present invention will now be described, by way of example only, with reference to the accompanying drawings, in which:

FIG. 1. shows a stimulus waveform and spectrogram of a 2 kHz pure-tone (PT) (left) and multi-tone (MT) stimuli (right). Stimuli are 50 ms in duration with a 10 ms rise and fall time.

FIG. 2a) shows grand average CAEP waveforms (n=15) generated by four pure-tone (PT). Responses are presented for three presentation levels, +10 dB (thick dashed line), +20 dB (thin clashed line), +40 dB (thin solid line).

FIG. 2b) shows grand average CAEP waveforms (n=15) generated by four one-octave-band multi-tone (MT). Responses are presented for three presentation levels, +10 dB (thick dashed line), +20 dB (thin dashed line), +40 dB (thin solid line).

FIG. 2c) shows grand average CAEP waveforms (n=15) generated by two broadband MT and two one-octave narrowband noise (NBN) auditory stimuli. Responses are presented for three presentation levels, +10 dB (thick dashed line), +20 dB (thin dashed line), +40 dB (thin solid line).

FIG. 3. shows CAEP rms amplitudes, collapsed over electrode positions FCz and Cz, for 0.5, 1, 2 and 4 kHz MT and PT stimuli for three sensation levels +10 dB, +20 dB, +40 dB SL. Vertical lines represent standard deviations between participants.

FIG. 4. shows CAEP rms amplitudes, collapsed over electrode positions FCz and Cz, for 1 and 2 kHz PT, NBN and MT stimuli for three sensation levels +10 dB, +20 dB, +40 dB SL, Vertical lines represent standard deviations between participants.

FIG. 5. shows CAEP rms amplitudes, collapsed over electrode positions FCz and Cz, for 0.5 and 2 kHz one-octave MT and broadband (LF and HF) MT stimuli at three presentation levels +10 dB, +20 dB, +40 dB SL. Vertical lines represent standard deviations between participants.

FIG. 6. shows a representation of the objective detection measure of 15 subjects (i.e. one trace per subject), for PT (left panel) and MT (right panel) stimuli for a 1 kHz center-frequency at +10 dB SL. The results of the Hotelling's T² which are calculated every 4 s are converted into z-score and presented across time. For z-score lower than −1.64 (i.e. p<0.05), the CAEP is considered to be present, assuming that the z score is compared to this threshold value at just one pre-specified point within each trace.

FIG. 7. shows cumulative CAEP detection z-scores, collapsed over electrode positions FCz and Cz, for 0.5, 1, 2 and 4 kHz MT (bold dashed line) and PT stimuli (solid line) for three sensation levels, +10 dB, +20 dB, +40 dB SL. Vertical lines represent standard deviations between participants.

FIG. 8. shows a stimulus representing the 1 kHz stimulus waveform and spectrogram representing the 3 sinusoids of frequency 793, 1000 and 1259 Hz.

### DETAILED DESCRIPTION

Systems according to embodiments of the invention include means for presenting an auditory stimulus to a person in the form of headphones or the like, means for generating the auditory stimulus in the form of a suitably configured signal generator or computing means, and means for measuring the cortical response of the person such as by way of electrodes applied to the scalp of the person.

One example of an auditory stimulus signal is a signal with component parts having regions of high power intensity in the form of pure tones at frequencies of 707 Hz, 891 Hz, 1122 Hz and 1414 Hz. This stimulus has a stimulus bandwidth of one octave (from 707 Hz to 1414 Hz) surrounding a center frequency of interest of 1000 Hz (in this case uniformly surrounding it on a logarithmic frequency scale). Each pure tone is separated by frequency regions containing little or no signal power.

In other embodiments the regions of high intensity may be provided as narrow frequency bands.

Stimuli with the desired characteristics can be generated by one of several methods including:
The addition of several pure tone signals, with each pure tone component having its frequency at one of the desired frequencies at which the power is to be concentrated.
Amplitude modulation of a signal at the centre frequency by a signal with another repetition frequency.
Frequency modulation of a signal at the centre frequency by a signal with another repetition frequency.
Filtering a noise that has power at all frequencies within the stimulus bandwidth by a filter that attenuates power at several frequencies within the stimulus bandwidth.
Repeatedly adding to the original signal a time-delayed version of a noise signal that has power at ail frequencies within the stimulus bandwidth.

The operation of embodiments of the invention will now be described by providing details of a study which has been carried out in relation to the invention as follows:

Subjects

Fifteen normal-hearing test subjects (7 males and 8 females) ranging from 23 to 43 years of age were recruited for this study. None of the participants reported any history of neurological abnormalities. Written consent was obtained from participants and the study was approved and conducted under the ethical supervision of the Australian Hearing Human Research Ethics Committee. Participants received a small monetary compensation for taking part in the study.

Auditory Stimuli

Twelve auditory stimuli were generated in Matlab (Mathworks). They comprised four sinusoidal pure-tone (PT) with frequencies 0.5, 1, 2 and 4 kHz, four one-octave multi-tone (MT) stimuli with the same center frequencies, two broadband MT stimuli—the first covering the low frequencies (0.25 to 1 kHz) and the second covering the high frequencies (1.5 to 8 kHz)—and two one-octave narrow bands of noise (NBN) centered around 1 and 2 kHz. All stimuli were 50 ms in duration with 10 ms rise-fall times to minimize spectral splatter.
a. Multi-Tone Stimuli

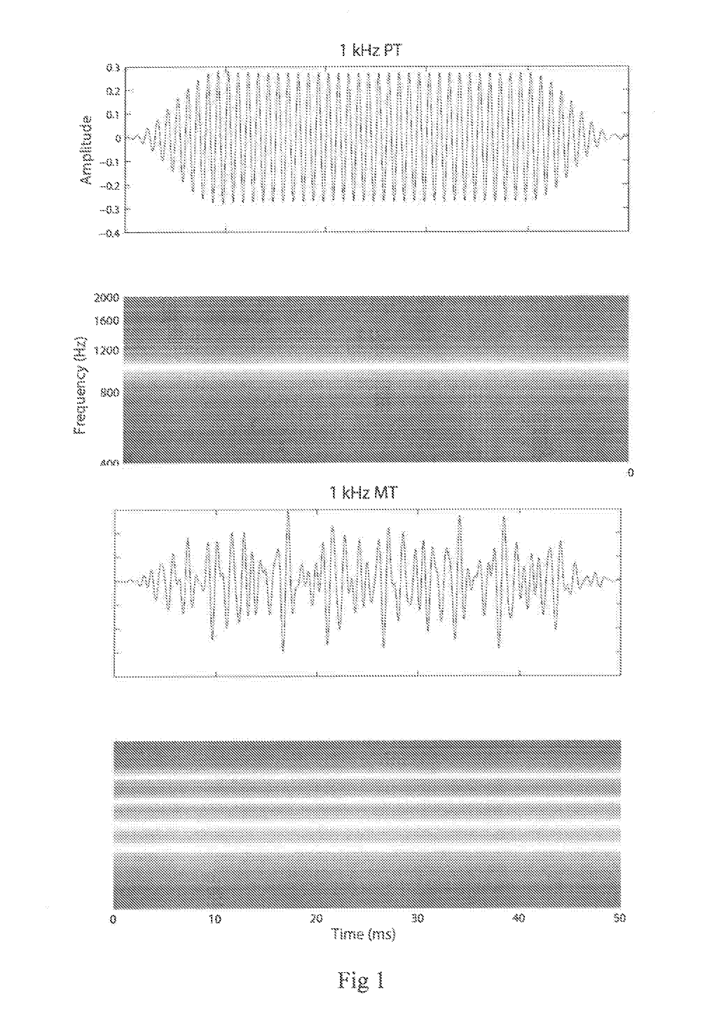

The MT stimuli were constructed by adding together a series of inharmonically related sinusoids. For the one-octave stimuli, the different tonal components were uniformly distributed around the center frequency on a logarithmic frequency scale. The sinusoids all had equal amplitude. For example, a MT stimulus with a center frequency of 1 kHz contained components with frequencies of 707, 891, 1122 and 1414 Hz with a stimulus bandwidth of one octave (front 707 Hz to 1414 Hz). The spectral characteristics for each stimulus are summarized in Table 1 while stimulus waveform and spectrogram are displayed in FIG. 1.

Calibration

All stimuli were acoustically calibrated at 70 dB HL according to the ISO standard 389-2 (ISO 1994) in an HA-2 2-cc coupler, incorporating a 1-inch 4144 microphone, a 1-to-½ inch DB0375 adaptor, and a 4230 sound level meter (all Brüel & Kjaer). Continuous stimuli were used for the calibration of tone-bursts, pure-tones and multi-tones.

Behavioral Procedure

Automatic Threshold Estimation

Test parameters of the computerized audiometry implemented using an adaptive staircase were based on Convery, E., Keidser, G., Seeto, M., et al. (2014) Identification of Conductive Hearing Loss Using Air Conduction Tests Alone: Reliability and Validity of an Automatic Test Battery. Ear and Hearing, 35, e1-e8. The SOAs, representing the time interval between the onset of two auditory stimuli, were of random duration and ranged from 1000 to 4600 ms. Participants were instructed to respond to the stimuli by pressing a button on a numeric keypad, A response was considered valid if it occurred within a 1.5-second time window commencing from the onset of the stimulus. The test included 3 phases, using a threshold seeking algorithm. The start level of stimulus presentation was 50 dB SPL. In phase 1, a 10-dB up/down step size was implemented. Phase 1 ended when the first non-response succeeding a positive response to a stimulus presentation was recorded. At this point the staircase “reversed” and intensity was increased by 10-dB prior to the next phase. Phase 2 used a 5-dB up/down step size. A subsequent non-response resulted in an increase in stimulus level in 5-dB increments until a positive response was recorded. After two reversals, a non-response resulted in a 5 dB increase for the next phase. In phase 3, the step size was lowered to 2 dB. Phase 3 ended when four reversals were recorded. A trimmed mean (i.e., removal of the highest and lowest values before averaging the remaining values) of all the presentations in phase 3 was calculated to determine the threshold. This threshold will be referred to as 0 dB SL (sensation level).

Behavioral Assessment

Participants underwent a series of audiometric assessments in a sound attenuated booth, to develop local normative data:
(1) Automatic pure-tone air conduction audiometry in both ears using stimuli 500 ms in duration with frequencies 0.25-8 kHz. The order of presentation of the pure-tones was 1, 2, 4, 8, 0.5, and 0.25 kHz. Stimuli were presented first in the right ear. Hearing thresholds had to be better than 20 dB HL in both ears to continue the test.
(2) One ear was selected pseudo-randomly such that 7 left and 8 right ears were used in the experiment (N=15 ears). Automatic air conduction audiometry was conducted using the twelve 50-ms auditory stimuli described in section “Auditory stimuli”. The presentation order of the twelve stimuli was randomized.

The thresholds obtained in (1) and (2) allow the difference (in dB) between the 500-ms long and 50-ms short, stimuli due to temporal integration (Moore 2012) to be estimated. All stimuli originated from .wav files stored on a desktop computer and were presented via a RME sound card (Fireface 800). All stimuli were delivered to the test ear through an ER-3A insert earphone (Etymotic Research).

Electrophysiological Recording of CAEPs

Sequence Generation

Sound sequences used for electrophysiological recording were generated for each participant based on their behavioral thresholds. The twelve stimuli described in section “Auditory stimuli” were presented at three sensations levels (+10, +20, +40 dB SL). Consequently, the total number of conditions in the experiment was 36. Stimulus conditions were randomized such that a full set of 36 stimulus conditions had to be presented before re-iteration. SOAs were jittered uniformly between 1000 and 3000 ms. Each condition was presented 60 times resulting in 2160 trials and a testing time of 72 minutes. MATLAB was utilized to create the sequence file.

Stimulus Presentation

The equipment from the behavioral experiment was used in the electrophysiological experiment to present the auditory stimuli. The stimuli were presented monaurally on the selected ear. An earplug was fitted to the opposite ear.

Data Acquisition

The electroencephalogram (EEC) was obtained with Neuroscan Synamps2 version: 4.3 (Compumedics) by placing four gold-plated electrodes onto the subject's head. Active electrodes were placed at fronto-central midline positions (Cz and FCz). The reference electrode was placed on the mastoid contralateral to the test ear, and the forehead (Fpz) acted as ground electrode (AES 1991). Prior to the placement of electrodes, the subject's skin was prepared using NuPrep EEG abrasive skin prepping gel. Water-soluble electrode paste was used to ensure a good connection between the electrodes and skin to achieve impedances of less than 5 kOhm across all electrode sites. Testing was conducted in an audiometric booth adhering to ANSI standard S.3.1-1999. During testing, the subjects were sitting comfortably in a dimmed, sound attenuated booth. The participants watched a muted close-captioned DVD of their choice which effectively captures attention without interfering with auditory processing. Participants were instructed not to pay attention to the stimulus.

Data Analysis

Amplitude measurements were analyzed at both FCz and Cz referenced to the mastoid contralateral to the test ear. All EEG channels were amplified with a gain of 2010, digitized at a sampling rate of 1000 Hz, and online bandpass filtered between 0.01 and 30 Hz. All epoched files were exported to MATLAB for off-line processing. The signal processing of the raw EEG files was partly conducted using EEGLAB (Delorme et al. 2003). An epoch of 700 ms (100 ms pre- and 600 ms post-stimulus onset) was used with baseline correction. Artifact and eye-blink were monitored by excluding epochs in excess of ±75 μV. A minimum of 52 accepted epochs was required for each stimulus condition.

Response Amplitude

Using the grand averages of the epoched waveforms, the “signal+noise” amplitude was expressed as the root mean square (rms) value within a window of 250 ms beginning 30 ms after stimulus onset. Due to the non-homogeneity of the variance across stimuli conditions and the dependence of the standard deviation on the mean response amplitude a log transform was applied on the amplitude data prior to statistical analysis, to stabilize the variance across conditions (Zacharias et al. 2011).

Measure of Response Detection

The Hotelling's T² statistic was used to provide an objective measure of CAEP response presence. Before applying the detection method, each recorded epoch was reduced to 9 averaged voltage levels, covering the range from 51 to 347 ms, with each bin being 33 ms wide. The bin width and number of bins were chosen based on earlier data (Golding et al. 2009), Response detection was based on the p-value obtained from a one-sample Hotelling's T² test on the bin-averaged data. The one-sample Hotelling's T² test is the multivariate extension of the ordinary one-sample t-test; instead of testing a null hypothesis that a scalar true mean equals a specified value, the Hotelling's T² test takes vector data and tests a null hypothesis that the true mean vector equals the zero vector. For every testing condition, the p-value was calculated after the collection of 9 epochs and subsequently, every additional two epochs. As the average SOA was 2 s, the p-value versus testing time could be presented for every subject. The p-values were afterwards converted into z-scores (assuming a normative z distribution) and a measure of response detection was calculated by cumulative summation of the z-score values. As two conditions (MT versus PT stimuli) were compared using the same sequential statistical testing, and no detection sensitivity was evaluated, no multiple testing adjustments needed to be performed.

Statistical Analysis

Repeated Measures ANOVAs

For statistical analysis, a three-way repeated measures analysis of variance (ANOVA) was performed on the log-transformed rms amplitudes and the measures of response detection. Greenhouse-Geisser corrections for sphericity were applied, as indicated by the cited ε value. Post-hoc comparisons were calculated using Tukey's test. Statistical analyses were conducted using Statistics 7.1 (StatSoft, Inc.) and R (R Development Core Team 2013), with the additional packages car (Fox et al, 2012), reshape (Wickham 2011), nlme (Pinheiro et al. 2013), and multcomp (Hothorn et al. 2013).

Results

Behavioral Thresholds

Table 2 presents the behavioral mean thresholds and standard deviations (in dB SPL) across 15 subjects for six 500-ms audiometric pure-tones (250-8000 Hz) and twelve 50-ms auditory stimuli. The mean threshold differences (in dB) across all subjects between 500-ms pure-tones and 50-ms tone-bursts for the frequencies 500, 1000, 2000 and 4000 Hz are shown in Table 2, The mean reaction time over all stimulus condition was 0.56 s. (SD =0.21). As expected the 50-ms tone-bursts had elevated thresholds when compared to 500-ms pure-tone thresholds. The mean behavioral threshold differences for the four tested frequencies ranged between 5 and 9 dB. The average threshold differences between 50-ms tone-bursts and 50-ms multi-tone stimuli ranged between 0 and 9 dB. These results can be used as corrections to account for the difference between the behavioral hearing thresholds estimated using 500-ms pure-tones and 50-ms tone-bursts.

Grand Average CAEP Waveforms

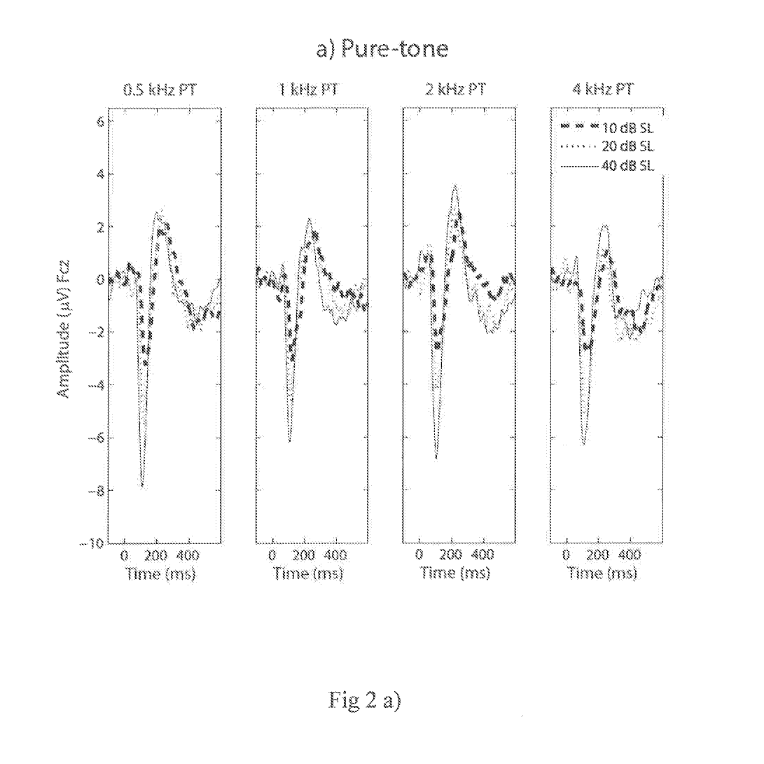

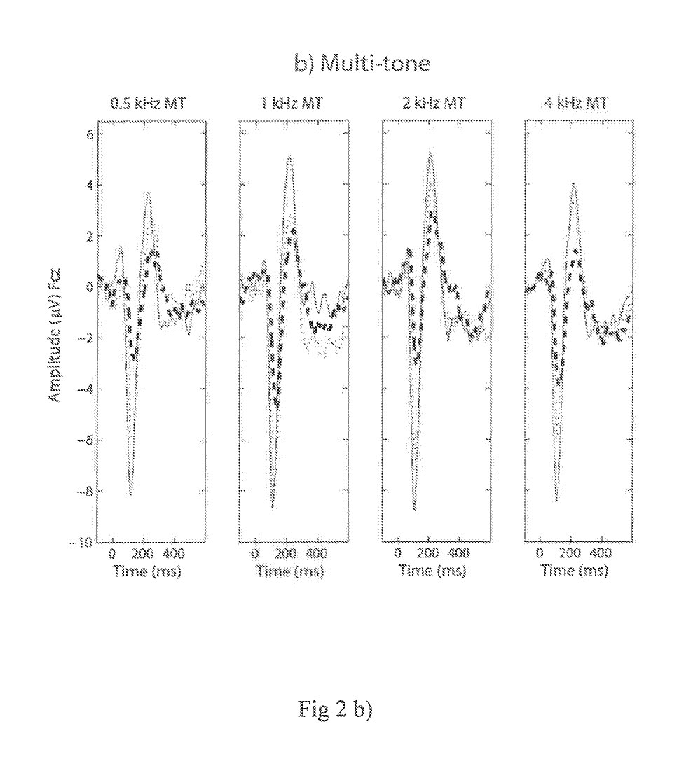

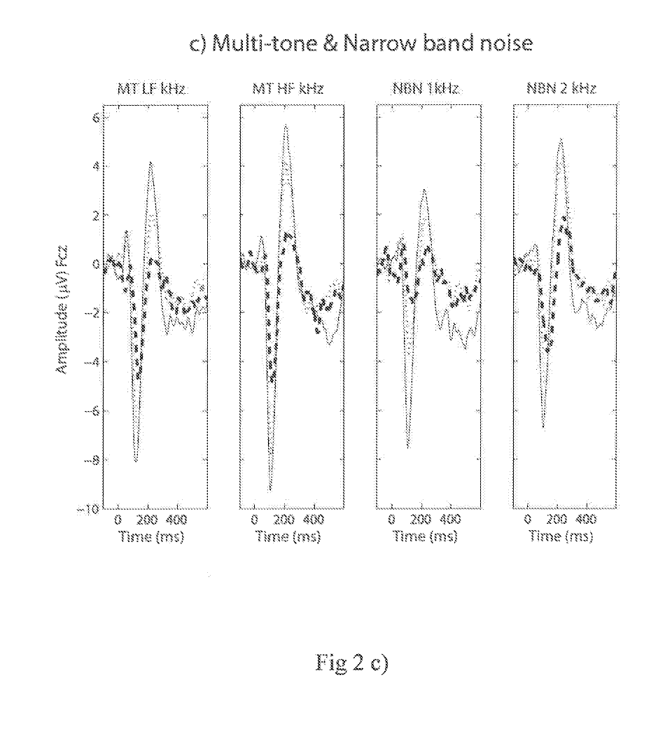

FIGS. 2a), 2b), 2c) show the mean CAEP waveforms, averaged across all fifteen subjects, in response to tone-bursts, one-octave-band multi-tone stimuli, broadband multi-tone stimuli and one octave-band noise, all 50 ms long and presented at +10, +20 and +40 dB SL. Clear CAEPs characterized by the P1-N1-P2 complex are identifiable by visual inspection for all conditions.

CAEP Amplitudes

PT Versus MT Stimuli

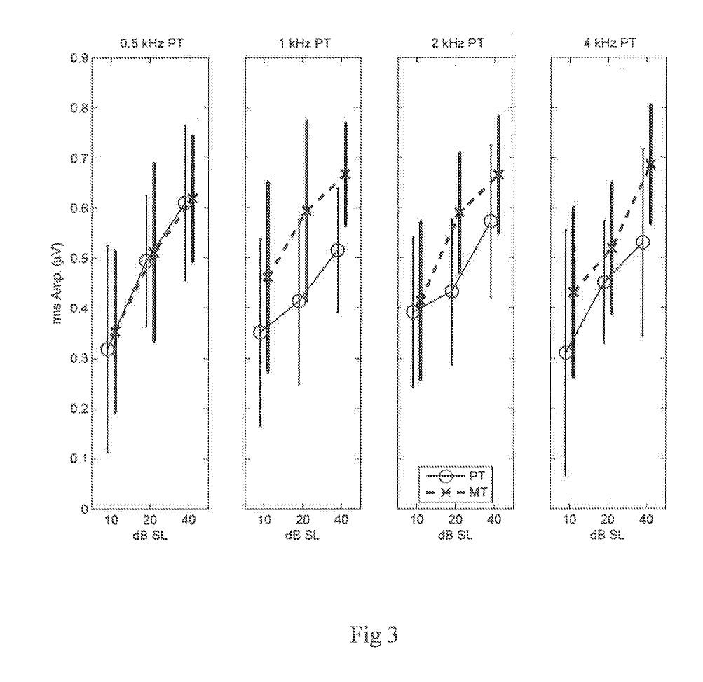

FIG. 3 summarizes the CAEP rms amplitudes in the time window 30-280 ms after stimulus onset as a function of stimulus (PT, one-octave MT stimuli), frequency (500, 1000, 2000 and 4000 Hz) and sensation level (10, 20, and 40 dB SL), while collapsed over EEG channels (Cz and FCz). A 2×2×4×3 repeated-measures ANOVA with EEG channel, stimulus, frequency and sensation level was performed on the rms amplitude data.

Effects of Stimuli (PT versus M 280 T) and Frequency (0.5, 1, 2 and 4 kHz)

The repeated-measures ANOVA revealed a main effect of stimulus (F(1,14)=67.36; p=0.000001; ε=1). The MT stimuli elicited significantly higher response amplitudes than PT. Moreover, an interaction effect was found between stimulus and frequency (F(3,42)=4.56; p=0.01; ε=0.01). Tukey pair wise comparisons showed no significant difference between PT and MT stimuli for 0.5 kHz (p=0.99) while significant differences were found for the other frequencies (i.e. 1, 2 and 4 kHz) (p<0.05). Table 3 shows the rms amplitude ratio between MT and PT stimuli and the time reduction (in %) to achieve the same SNR for MTs as PTs. Time reduction is calculated based on the MT/PT ratio, assuming that the residual noise in the averaged waveform decreases with the square root of the number of epochs. When collapsing the data across the three frequencies 1, 2 and 4 kHz and all levels, an average rms amplitude ratio of 1.32 (95 % confidence interval 1.25-1.37) was found for MT stimuli when compared to PT, which corresponds to a potential 46 % average time reduction.

Effect of Sensation Level (10, 20 and 40 dB SL)

A main effect of sensation level was found (F(2,28)=122.66; p<0.000001; ε=0.86) with higher intensities eliciting larger CAEP amplitudes.

Effect of Channel (FCz Versus Cz)

A main effect of channel was found (F(1,14)=17.74; p=0.0008; ε=1) with a 11% rise of rms amplitudes obtained from channel FCz-mastoid than from Cz-mastoid (95% confidence interval 7-14%).

NBN Versus PT and MT Stimuli at Frequencies 1 and 2 kHz

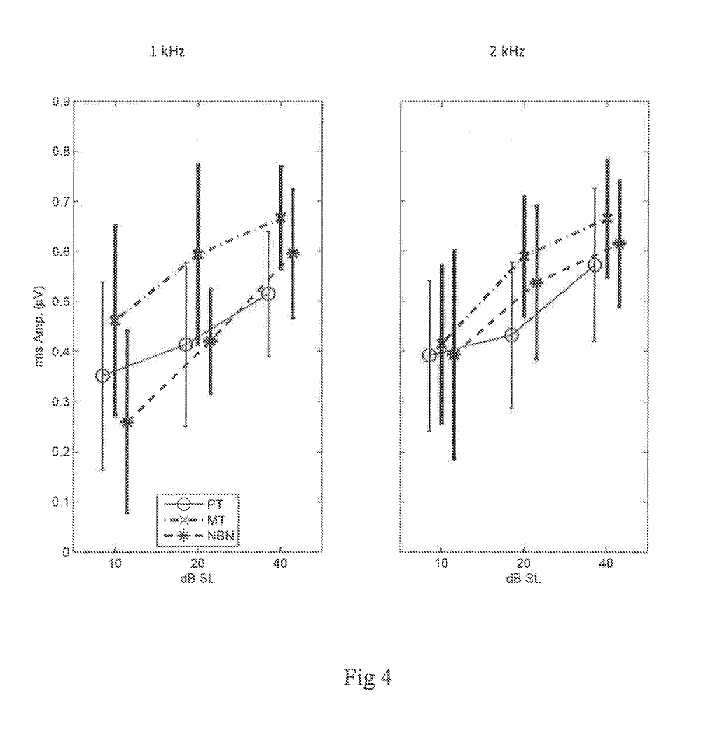

Rms amplitudes of the CAEP elicited by NBN were compared to responses of both MT and PT stimuli in a 2×3×2×3 repeated-measures ANOVA with channel, stimulus, frequency and level. FIG. 4 shows rms CAEP amplitudes as a function of stimulus (i.e. PT, one-octave MT stimulus, and one-octave NBN), for the two frequencies (1000 and 2000 Hz) and the stimulus level (+10, +20, and +40 dB SL). Of interest, a main stimulus effect was found (F(2,28)=26.23; p=0.000003; ε=0.92). Tukey pairwise comparisons revealed no significant difference between PT and NBN (p=0.37) but a significant difference between MT stimuli and both NBN and PT (p < 0.001). A significant interaction between stimulus and frequency was present (F(3,42)=4.55; p=0.01; ε=1). That is, the effect of stimulus is larger at 1 kHz than at 2 kHz.

One-Octave (0.5 and 2 kHz) Versus Broadband (UF and HF) MX Stimuli

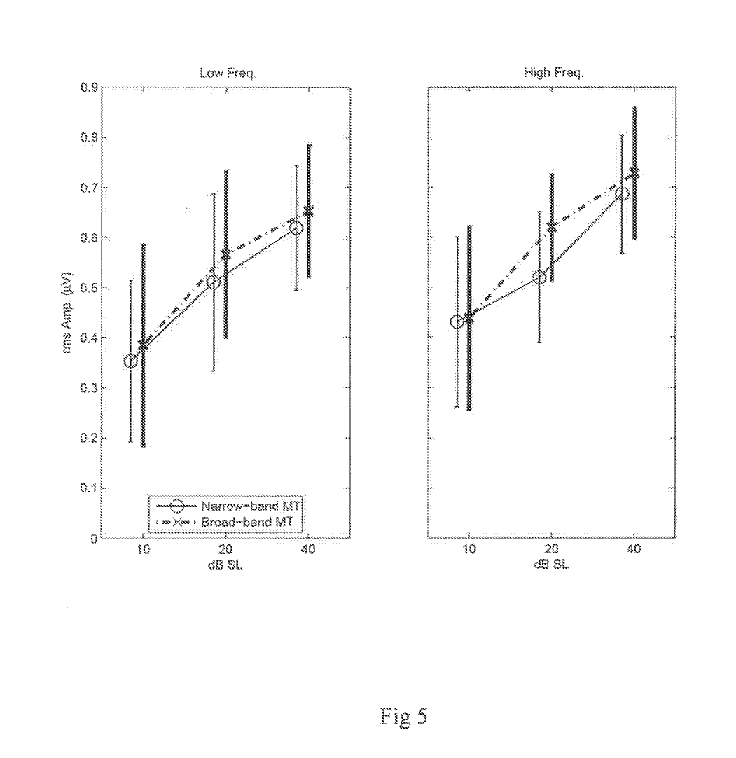

FIG. 5 shows rms CAEP amplitudes for one-octave and broadband MT stimuli. Although the mean rms amplitude for the broadband MT stimuli was larger in every condition, a one-way repeated-measures ANOVA did not show a significant difference between the two stimuli (F(1,14)=2.65: p=0.12; ε=1).

Objective Detection Scores of the CAEP

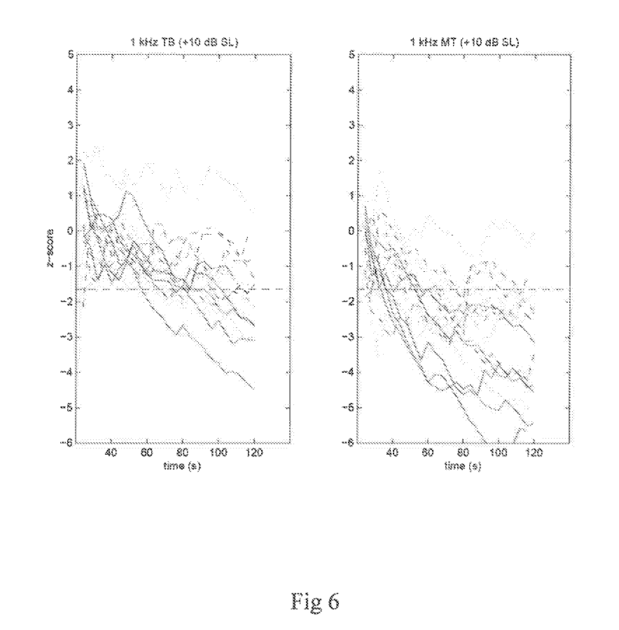

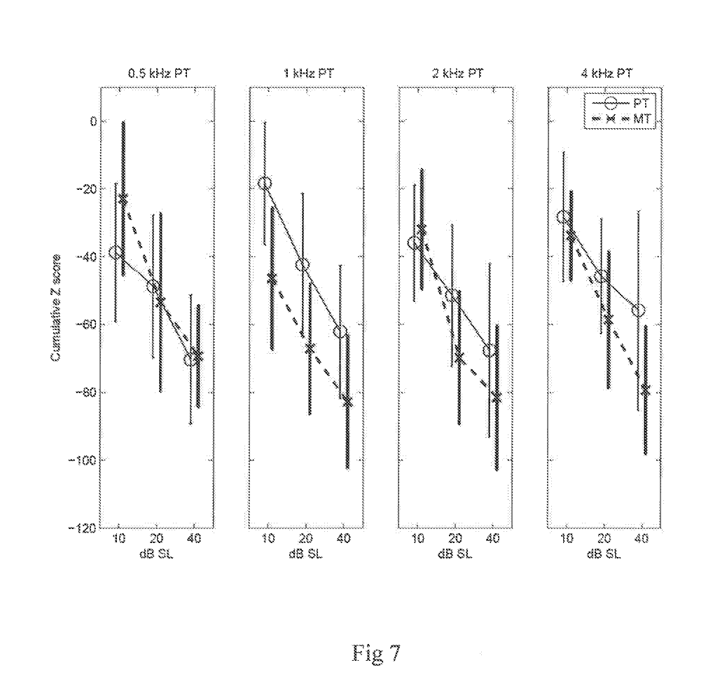

FIG. 6 shows an example of the representation of z-score traces for the tone-burst (left) and multi-tone stimulus (right) for the fifteen subjects. A more negative z-score represents a smaller p value, and therefore a higher response detection. Hence, in this example, the response to the multi-tone stimulus is more likely to be objectively detected than to the toneburst. It is valuable to have this measure as the Hotelling's T² is clinically used for the detection of cortical responses. Mean cumulative z-scores are displayed in FIG. 7 for all stimuli, frequencies and sensation levels. Once again, more negative cumulative z-scores translate into higher detections of the responses. A 2×2×4×3 repeated-measures ANOVA with EEG channel, stimulus, frequency and sensation level was performed to assess their effects on the z-score data. It revealed a main effect of stimulus (F(1,14)=41.22; p=0,00001; ε=1). Significantly more negative mean cumulative z-scores for the MT stimuli were observed when compared to z-scores from PT. A main effect of level was observed as well (F(2,28)=100.70; p<0.00001; ε=0.79) with higher sensation levels showing significantly more negative z-scores. No main effect of channel was observed, indicating no advantage for a specific channel (i.e. FCz-M versus Cz-M) (F(1,14)=0.60; p=0.45; ε=1.00). This is in contrast with the main channel effect for CAEP amplitudes, which indicated significantly larger amplitudes at FCz. This is likely caused by increased noise at this electrode position,

A significant interaction between stimulus and level (F(2,28)=4.70; p=0.02; ε=1) was observed. Tukey pairwise comparisons indicated no difference in z-scores between PT and MT stimuli for +10 dB SL (p=0.85) while significant differences were found for the other levels (i.e. +20 and +40 dB SL) (p<0.001). A significant interaction was present between stimulus and frequency (F(3,42)=13.23; p<0.00001; ε=0.82). Similarly to the CAEP amplitudes, Tukey pairwise comparisons revealed a significant effect of the stimulus for the frequencies 1, 2 and 4 kHz (p <0.001) but not for 0.5 kHz (p=0.22).

Discussion

In the present study, we designed narrowband multi-tone (MT) stimuli centered around 0.5, 1, 2 and 4 kHz. We compared the cortical auditory evoked potentials (CAEPs) they elicited with responses to sinusoidal pure-tone (PT), one-octave, narrow-band noise (NBN), and one octave, multi-tone complexes, in total, electrophysiological responses were recorded for 12 different stimuli at 3 sensation levels (+10, +20 and +40 dB SL) for which clear P1-N1-P2 waveforms could be discerned. In a group of subjects with normal hearing it was found that the amplitude of the CAEP was influenced by the spectral composition of the auditory stimuli, with all auditory stimuli matched in sensation level. First, the effect of stimulus will be discussed by comparing the CAEP amplitudes of both the MT and the NBN stimulus with those of the PT. The latter group served as the reference. Second, we reflect on the physiological reasons underlying the change of the cortical response characteristics. Finally, the potential benefit of using MT stimuli for threshold estimation In a clinical setup is considered.

PT Versus MT Stimuli

Responses elicited by MT stimuli with frequencies centered around 1, 2 and 4 kHz showed a significantly larger rms CAEP amplitude than responses to PT stimuli. These results show that, the neural response to a pure tone is different from that to a complex tone centered on the same frequency.

FT Versus NBN Stimuli

The inclusion of two NBN stimuli at 1 and 2 kHz in the experimental design allowed investigation as to whether the growth of the cortical response was driven by the frequency bandwidth or by the arrangement of the frequency components i.e. spectral line structure. The main effects in FIG. 4 showed no significant amplitude differences for CAEPs elicited by NBN and PT stimuli. Conversely, significantly larger amplitudes were observed for MT stimuli when compared to both PT and NBN. This suggests that the spectral fine structure of the sound, rather than its bandwidth, is principally affecting the cortical response, This observation is reinforced by the results in FIG. 5, which showed no significant main differences between one-octave and multi-octave MT stimuli. A limitation of the present study is that the small sample size could be the factor explaining the lack of any significant difference between the two types of MT stimuli. However, the observed small effect size makes any differences clinically unimportant.

Possible Functional Reasons

Without wishing to be bound by theory, the inventors offer the following discussion of the possible reasons why the invention works.

There are at least four possible reasons why complex stimuli may elicit larger responses than pure tones. First, the tonotopic arrangement of the auditory system, including the primary auditory cortex (Howard III, M. A., Volkov, I. O., Abbas, P. J., et al. (1996). A chronic microelectrode investigation of the tonotopic organization of human auditory cortex. Brain Research, 724, 260-264) means that stimuli with wider bandwidths may evoke cortical activity in a more widespread group of neurons immediately surrounding those that respond best to pure tones at the centre frequency. If the total number of neurons increases, so too may the magnitude of the cortical responses. This would be analogous to the way that, for sounds at moderate input levels, loudness increases with bandwidth when total intensity is held constant.

Second, rather than a larger number of neuronal firings, the MT stimulus could somehow cause the same neurons to fire more synchronously with each other, which by itself would increase the magnitude of the cortical response on the scalp.

Third, the MT stimulus may excite neuron firing in cortical regions remote from those excited by a pure tone. Functional magnetic resonance imaging (fMRI) studies found that the complexity of the auditory stimulus has an effect on the area of activation in the auditory.

Fourth, a complex spectrum where frequency regions of high intensity alternate with regions of low intensity (i.e. a line spectrum, whether harmonically or inharmonically related) may give rise to complex excitatory and especially inhibitory stimulation between adjacent tonotopic regions within the cortex. Such interactions may occur to a much lesser degree with stimuli that have a more diffuse spectrum, even when the two stimuli extend over the same total bandwidth.

Of these four possibilities, the fourth and possibly the third are the most consistent with the data in this experiment. The first reason (more locally extensive activity as a result of increased bandwidth) cannot be responsible. This follows because of the lack of difference between the response to PT and NBN (FIG. 3), the significant difference between the response to MT stimuli and NBN of the same bandwidth (FIG. 4), and the lack of difference in the response to narrow band and wide-band MT (FIG. 5). Increased bandwidth therefore seems not to be the feature of the stimulus that causes a larger response with complex stimuli, so we can reject the idea, that the increased amplitude comes just from locally enlarging the response region of auditory cortex in a manner tonotopically related to stimulus bandwidth.

Although we certainly cannot rule out the second reason, we cannot identify any temporal feature in the MT stimulus that seems capable of inducing greater synchronicity of firing. Because the components of the MT stimulus are inharmonically related, the phase relationship between each pair of components within the set is constantly changing. The only temporal aspect they have in common is their onset and offset, and it is difficult to see why the same set of neurons would respond more synchronously to the onset of the MT stimulus than they do to the onset of the pure tone stimulus.

The third explanation, more remotely extensive neuron firing for the MT stimulus, seems possible. If so, again it is certainly not just, the increased bandwidth of the MT stimuli that induces the more widespread remote activity, as the amplitude increase did not occur for the NBN stimuli.

Potential Benefit of Using MT Stimuli in a Clinical Setup

CAEPs are increasingly used in clinical applications for both hearing aid evaluation and hearing loss diagnostics. As a result, reduction of measurement time is of great interest. An advantage of stimuli that elicit larger CAEP responses is a reduction of the number of averages required to extract the response from background noise, resulting in a shorter test duration (see table 3). The use of frequency-specific MT stimuli may therefore be of clinical use in. assessing hearing thresholds objectively.

Corrections Due to Temporal Integration

Auditory stimuli used for CAEP recording are generally shorter than those used for behavioral assessment, due to optimal stimulus lengths for CAEP recording being up to 70 ms. As stimulus duration lengthens, the perceived loudness of a sound increases and detection threshold lowers (Moore 2012). In this case, it is important to apply corrections to compensate for the higher thresholds found when using short duration stimuli.

The results from the behavioral aspect of this study allowed determination of these corrections, which account for the difference between hearing thresholds for long and short stimuli due to temporal integration. These values were provided in Table 2. The mean behavioral threshold differences between 50-ms short and 500-ms long stimuli ranged from 5 to 9 dB. It is important to account for these differences in order to determine behavioral hearing thresholds and optimize subsequent hearing aid fitting.

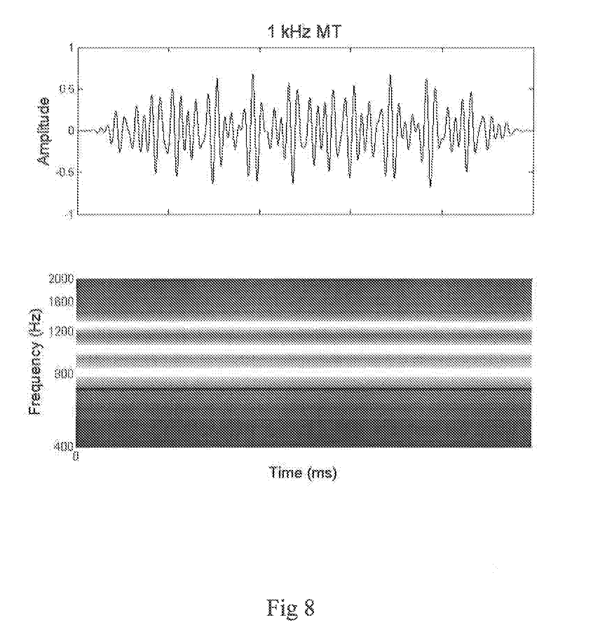

In the study described above, the narrow-band multi-tone (MT) stimuli were one-octave wide and composed of 4 tonal components. In other embodiments, the stimulus may take other forms. In one embodiment, the multi-tone (MT) stimuli are an inharmonic ⅔ octave wide, composed of 3 tonal components logarithmically spaced and centered around the center frequency. FIG. 8 shows such a stimulus by way of a graph representing the 1 kHz stimulus waveform and spectrogram representing the 3 sinusoids of frequency 793, 1000 and 1259 Hz.

Embodiments of the invention reduce the time taken to perform hearing threshold testing thus saving time costs in engaging a professional audiologist and improving patient comfort by reducing the time a patient must spend in testing.

In this specification the expression “a region of low intensity” includes a region in a signal with substantially zero or no power as well as regions with some power.

Any reference to prior art contained herein is not to be taken as an admission that the information is common general knowledge, unless otherwise indicated.

Finally, it is to be appreciated that various alterations or additions may be made to the parts previously described without departing from the spirit or ambit, of the present invention.

TABLE 1

| Center | | | | | | | | |
|---|---|---|---|---|---|---|---|---|
| Frequency | Frequency (in Hz) of each sinsoidal component | | | | | | | |
| 0.5 kHz | 353 | 445 | 561 | 707 | | | | |
| 1 kHz | 707 | 890 | 1122 | 1414 | | | | |
| 2 kHz | 1414 | 1781 | 2244 | 2828 | | | | |
| 4 kHz | 2828 | 3563 | 4489 | 5656 | | | | |
| Low Freq. | 250 | 315 | 397 | 500 | 630 | 794 | 1000 | |
| High Freq. | 1500 | 1889 | 2381 | 3000 | 3779 | 4762 | 6000 | 7559 |

### Table 1. Frequency Content of Multi-Tone Stimuli:

TABLE 2

| | | | | Threshold difference | | |
|---|---|---|---|---|---|---|
| | | Threshold | RET 0 dB HL | PT − PT₅₀₀ | MT − PT₅₀₀ | MT − PT |
| Stimuli | | (dB SPL) | (dB SPL) | (dB) | (dB) | (dB) |
| PT₅₀₀ 0.25 | kHz | 18.7 ± 5.9 | 14 | | | |
| PT₅₀₀ 0.5 | kHz | 7.4 ± 5.1 | 5.5 | | | |
| PT₅₀₀ 1 | kHz | 0.3 ± 5.6 | 0 | | | |
| PT₅₀₀ 2 | kHz | 9.7 ± 4.2 | 3 | | | |
| PT₅₀₀ 4 | kHz | 4.8 ± 6.8 | 5.5 | | | |
| PT₅₀₀ 8 | kHz | −1.0 ± 8.2 | 0 | | | |
| PT 0.5 | kHz | 15.8 ± 6.0 | | 8.4 ± 4.4 | | |
| PT 1 | kHz | 9.3 ± 4.0 | | 9.0 ± 4.4 | | |
| PT 2 | kHz | 14.9 ± 3.5 | | 5.1 ± 2.2 | | |
| PT 4 | kHz | 9.9 ± 6.2 | | 5.1 ± 3.8 | | |
| MT 0.5 | kHz | 17.9 ± 4.3 | | | 10.5 ± 4.0 | 2.1 ± 4.5 |
| MT 1 | kHz | 12.1 ± 3.2 | | | 11.8 ± 5.0 | 2.8 ± 3.7 |
| MT 2 | kHz | 15.0 ± 3.9 | | | 5.3 ± 4.0 | 0.1 ± 3.3 |
| MT 4 | kHz | 18.5 ± 5.1 | | | 13.7 ± 8.2 | 8.6 ± 5.7 |
| LF MT | | 14.8 ± 3.1 | | | | |
| HF MT | | 16.1 ± 4.5 | | | | |
| NBN 1 | kHz | 12.4 ± 3.0 | | | | |
| NBN 2 | kHz | 16.5 ± 3.2 | | | | |

Table 2. Behavioral mean thresholds and standard deviations across 1S subjects for six 500-ms pure-tones (PT500) (0.25-8 kHz), and twelve 50-ms auditory stimuli used for the recording of CAEPs. The twelve stimuli consisted of four PT with frequencies 0.5, 1, 2 and 4 kHz, four band-limited (one-octave) multi-tone (MT) stimuli with the same center frequencies, two broadband MT stimuli covering the low (LF MT: 0.25 to 1 kHz) and high frequencies (HF MT: 1.5 to 8 kHz) and two one-octave narrow bands of noise (NBN) centered around 1 and 2 kHz. Use reference equivalent threshold (RET, i.e. 0 dB HL) according to ISO Standardization (1994) is provided. The mean threshold difference and standard deviation between 50 and 500 ms PT, between MT and PT₅₀₀ and between MT and 50 ms PT are provided.

TABLE 3

| Frequency | Level | Rms amplitude ratio | Estimated time | |
|---|---|---|---|---|
| (kHz) | (dB SL) | MT/PT | reduction (%) | p-value |
| 0.5 | 10 | 1.08 (0.88:1.33) | 14.7 | 0.97 |
| | 20 | 1.04 (0.85:1.27) | 7.4 | 1.00 |
| | 40 | 1.02 (0.83:1.25) | 4.1 | 1.00 |
| 1 | 10 | 1.29 (1.05:1.58) | 39.8 | 0.004 |
| | 20 | 1.51 (1.23:1.85) | 56.3 | <0.0001 |
| | 40 | 1.42 (1.16:1.74) | 50.2 | <0.0001 |
| 2 | 10 | 1.05 (0.86:1.29) | 9.8 | 1.00 |
| | 20 | 1.44 (1.17:1.76) | 51.5 | <0.0001 |
| | 40 | 1.24 (1.01:1.52) | 34.8 | 0.03 |
| 4 | 10 | 1.32 (1.07:1.62) | 42.4 | 0.001 |
| | 20 | 1.17 (0.96:1.44) | 27.1 | 0.28 |
| | 40 | 1.43 (1.17:1.76) | 51.2 | <0.0001 |

Table 3: mean and 95% confidence intervals of the rms amplitude ratio MT/PT at 10, 20 and 40 dB SL for stimuli with center frequencies at 0.5, 1, 2 and 4 kHz. An estimation of the time reduction using MTs when compared to PTs to reach a similar SNR is provided. The last column slows a p-value calculated using a mixed-effects model. It displays whether the difference between MT and PT stimuli is significant.

1-16. (canceled)

17. A method of determining a hearing threshold of a person, the method comprising:
presenting an auditory stimulus of a duration of less than one second to the person;
spreading a power of the stimulus across a frequency range in alternating regions of high and low intensity;
including at least two regions of high intensity in the stimulus; and
measuring a transient evoked electrical response of a brain of the person.

18. The method according to claim 17, further comprising centering the at least two alternating regions of high and low intensity around a target frequency of interest.

19. The method according to claim 17, further comprising including at least three regions of high intensity in the stimulus.

20. The method according to claim 17, further comprising including narrow bands in the regions of high intensity.

21. The method according to claim 17, further comprising including pure tones in the regions of high intensity.

22. The method according to claim 17, further comprising presenting the stimulus to the person for a duration of less than 200 ms.

23. The method according to claim 17, further comprising presenting the stimulus to the person for a duration of less than 100 ms.

24. The method according to claim 17, further presenting the stimulus to the person for a duration of approximately 50 ms.

25. A system for determining hearing thresholds of a person, the system comprising:
means for presenting an auditory stimulus of a duration of less than one second to the person;
a power of the stimulus being spread across a frequency range in alternating regions of high and low intensity;
the stimulus including at least two regions of high intensity; and
means for measuring a transient evoked electrical response of a brain of the person.

26. The system according to claim 25, wherein the alternating regions of high and low intensity are centered around a target frequency of interest,

27. The system according to claim 25, wherein the stimulus includes at least three regions of high intensity,

28. The system according to claim 25, wherein the regions of high intensity include narrow bands.

29. The system according to claim 25, wherein the regions of high intensity include pure tones.

30. The system according to claim 25, wherein the stimulus has a duration of less than 200 ms.

31. The system according to claim 25, wherein the stimulus has a duration of less than 100 ms.

32. The system according to claim 25, wherein the stimulus has a duration of approximately 50 ms.
